(19) United States
(12) Patent Application Publication
(10) Pub. No.: US 2011/0045561 A1
(43) Pub. Date: Feb. 24, 2011

(54) Method of Stimulating Ethanol Production and Growth of Aquatic Plants

(76) Inventor: Tony A. Hagen, Sioux Falls, SD (US)

Correspondence Address: SEAN KAUFHOLD, P. O. BOX 89626, SIOUX FALLS, SD, 57109, US

(21) Appl. No.: 12/730,213

(22) Filed: Mar. 23, 2010

Related U.S. Application Data
(63) Continuation-in-part of application No. 12/437,333, filed on May 7, 2009.
Application No. 12/437,333 is a continuation-in-part of application No. 12/628,601, filed on Dec. 1, 2009.

(51) Int. Cl. C12P 7/06; C07C 31/08

(52) U.S. Cl. 435/161; 568/840

(57) ABSTRACT

A method of stimulating ethanol production and growth of aquatic plants includes the steps of placing aquatic plants in a cell containing water and creating an oxygenated condition within the cell to initiate an aerobic process. The aquatic plants create and store carbohydrates during the aerobic process. The cell is then covered with a light blocking cover during the anoxic condition to inhibit light from entering the cell. An anoxic condition is created within the cell to initiate an anaerobic process by the aquatic plants. The aquatic plants increase in size and release ethanol into the water by metabolism of stored carbohydrates during the anaerobic process. The ethanol is then sequestered from the water.

This application is a continuation in part of U.S. patent application Ser. No. 12/437,333 filed on May 7, 2009 and U.S. patent application Ser. No. 12/628,601 filed Dec. 1, 2009.

### BACKGROUND OF THE DISCLOSURE

### Field of the Disclosure

The disclosure relates to ethanol production methods and more particularly pertains to a new ethanol production method for promoting plant growth by plants which produce free ethanol during anaerobic metabolism to form a self-sustaining cycle of plant growth and ethanol production.

### SUMMARY OF THE DISCLOSURE

An embodiment of the disclosure meets the needs presented above by generally comprising the steps of placing aquatic plants in a cell containing water and creating an anoxic and dark condition within the cell to initiate an anaerobic process by the aquatic plants. The aquatic plants increase in size and release ethanol by metabolism of stored carbohydrates during the anaerobic process. A lighted condition is then created and oxygenation allowed within the cell to initiate an aerobic process. The aquatic plants create and store carbohydrates during the aerobic process. The steps of creating anoxic and oxygenated conditions are repeated to stimulate aquatic plant growth and the release of ethanol.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawing wherein:

FIG. 1 is a schematic view of a method of stimulating ethanol production and growth of aquatic plants according to an embodiment of the disclosure.

FIG. 2 is a schematic view of a method of stimulating ethanol production and growth of aquatic plants according to an embodiment of the disclosure.

FIG. 3 is a schematic view of a method of stimulating ethanol production and growth of aquatic plants according to an embodiment of the disclosure.

### DESCRIPTION OF THE PREFERRED EMBODIMENT

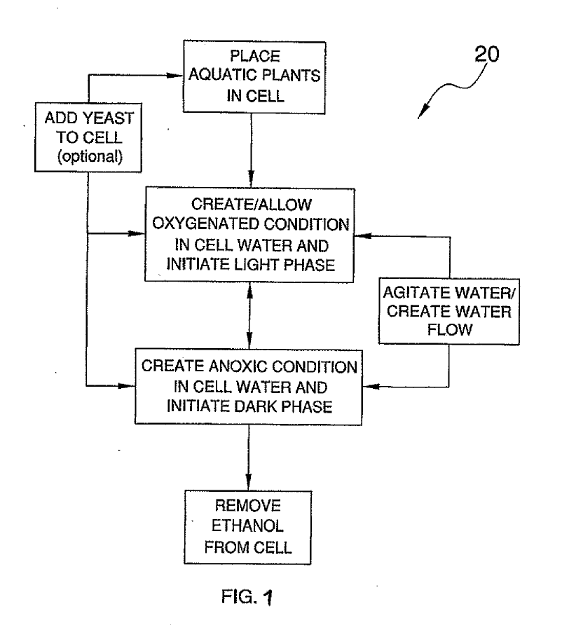

With reference now to the drawings, and in particular to FIGS. 1-3, a new ethanol production method embodying the principles and concepts of an embodiment of the disclosure and generally designated by the reference numeral 10 will be described.

As illustrated in FIGS. 1-3, the method 20 of stimulating ethanol production and growth of aquatic plants generally growing aquatic plants in a cell. The aquatic plants may be acquired in any conventional manner such as gathering them from lakes or ponds, growing them in growing tanks or growing them directly in the cell. As the method 20 is performed, it may be used to grow and provide aquatic plants as they are needed for future cells or for replacement purposes. The cell is constructed to hold water and may or may not be lined to prevent transfer of fluids and gases into a ground surface supporting the cell. A substrate, for example a fine particulate, may be placed in the cells and the aquatic plants introduced into the cells where they can anchor themselves in the particulate. A fine particulate may used as it may promote less energy expenditure on the part of the aquatic plants to root growth into the particulate and retains a higher percentage of the plant matter above the surface of the particulate. However, many of the plants being utilized by the method 20 primarily rely on their root systems as anchoring means and therefore any type of anchoring mechanism or substrate may be used which can be engaged by the roots. This may include mechanical anchoring devices, such as grids or screens, to which the roots may engage and couple themselves. Additionally, a denser particulate may be useful where water flow within the cell requires a stouter anchoring substrate.

It should be understood that the method may be practiced with a number of cells and the size a cell is not crucial to the method. The size of cell may be dictated by available land area, access to raw materials and cost controls, though it should be understood that the method may be practiced with only a single cell. The cell may have any depth required for the chosen aquatic plant to properly grow. While the cell may have often have a depth of between 10 cm and 7 m to prevent restricted plant growth, it has been found that some plants may grow in dramatically deeper depths providing other environmental factors, such as atypically high water temperatures at depth, are present. For instance, Stuckenia pectinate has been shown to grow in depths of greater than 20 m of water where thermal vents provide at least warmer water than would be typically found in a North American lake at such depths.

The cell may also be temperature controlled and in particular the cell should be prevented from freezing which may kill the aquatic plants. Heat for the cells may be sequestered from waste heat emitted by adjacent ethanol processing plants or any other convenient source of waste heat. Additional heat sources, such as geothermic and solar, may also be utilized where convenient. However, in particularly hot climates, the cells may require cooling to prevent temperatures that would otherwise harm the plants. Depending on the variety of aquatic plant being utilized, a temperature range may be selected which optimizes plant growth and ethanol production. For example, some selected plants such as Stuckenia pectinate may be maintained between 85° Fahrenheit and 73° Fahrenheit for optimal growth, though it should be understood that the overall temperature range for growth and production of ethanol falls into a much wider range. One manner of controlling temperature is to sink the cell into the ground where the soil around the cell will moderate the temperature of the cell.

The aquatic plants may be selected from any number of aquatic plants which readily live in or on an aquatic environment such as directly in water or in permanently saturated soil. More generally, the term “aquatic plant” may include any algae, aquatic plant or semi-aquatic plant which has a high tolerance for either being constantly submerged in water or intermittently submerged during periods of flooding. Further, more than one type of aquatic plant may be used within a single cell.

The aquatic plants may include, for example, algae, submersed aquatic herbs such as, but not limited to, Stuckenia pectinate (formerly known as Potamogeton pectinatus), Potamogeton crispus, Potamogeton distintcus, Potamoteton nodosus, Ruppia maitima, Myriophyllum spicatum, Hydrilla verticillata, Elodea densa, Hippuris vulgaris, Aponogeton boivinianus, Aponogeton rigidifolius, Aponogeton longiplumulosus, Didiplis diandra, Vesicularia dubyana, Hygrophilia augustifolia, Micranthemum umbrosum, Eichhornia azurea, Saururus cernuus, Cryptocoryne lingua, Hydrotriche hottoniiflora, Eustralis stellata, Vallisneria rubra, Hygrophila salicifolia, Cyperus helferi, Cryptocoryne petchii, Vallisneria americana, Vallisneria torta, Hydrotriche hottonilflora, Crassula helmsii, Limnophila sessiliflora, Potamogeton perfoliatus, Rotala wallichii, Cryptocoryne becketii, Blyxa aubertii and Hygrophila difformmis, duckweeds such as, but not limited to, Spirodela polyrrhiza, Wolffia globosa, Lemna trisulca, Lemna gibba, Lemna minor, and Landoltia punctata, water cabbage, such as but not limited to Pistia stratiotes, buttercups such as but not limited to Ranunculus, water caltrop such as but not limited to Trapa natans and Trapa bicornis, water lily such as Nymphaea lotus, Nymphaeaceae and Nelumbonaceae, water hyacinth such as but not limited to Eichhornia crassipes, Bolbitis heudelotii, and Cabomba, and seagrasses such as but not limited to Heteranthera zosterifolia, Posidoniaceae, Zosteraceae, Hydrocharitaceae, and Cymodoceaceae. Moreover, in one of the various embodiments, a host alga may be selected from the group consisting of green algae, red algae, brown algae, diatoms, marine algae, freshwater algae, unicellular algae, multicellular algae, seaweeds, cold-tolerant algal strains, heat-tolerant algal strains, ethanol-tolerant algal strains, and combinations thereof.

The aquatic plants in general may also be selected from one of the plant families which include Potamogetonaceae, Ceratophyllaceae, Haloragaceae, and Ruppiaceae. More particularly, the aquatic plants chosen should have a large Pasteur effect which increases the ratio of anaerobic CO₂ production to the aerobic CO₂ production. Typically this ratio is on the order of 1:3, but aquatic plants such as for example Stuckenia pectinata, formerly and also sometimes known as Potamogeton pectinatus, and commonly known as Sago Pondweed, may increase this ratio to 2:1 as explained in “Anoxia tolerance in the aquatic monocot Potamogeton pectinatus: absence of oxygen stimulates elongation in association with an usually large Pasteur effect,” Journal of Experimental Botany, Volume 51, Number 349, pp. 1413-1422, August 2000, which is incorporated herein by reference. During an elongation process which takes place in a dark and anoxic environment, the plant elongates to form cellular chambers which will later be used to store carbohydrates formed during aerobic metabolism through an aerobic process of the aquatic plant. These carbohydrates can then be used to release ethanol during anaerobic metabolism through an anaerobic process of the aquatic plant. Generally, the equations are as follows:

Aerobic plant metabolism: 6CO₂+6H₂O→6O₂+C₆H₁₂O₆

Anaerobic plant metabolism: C₆H₁₂O₆→2CO₂+2C₂H₅OH

Once the aquatic plants are established in a cell, the water in the cell is placed in an anoxic condition. This may be accomplished in several ways either by themselves or in combination with each other. For instance, anoxic water may be introduced into the cell or the oxygen may be severely depleted (i.e. rendered anoxic) from the water using organic or mechanical means. The term “anoxic” is here defined as the point of oxygen depletion that induces the plant to enter an anaerobic metabolic condition, as it should be understood that a very small quantity of oxygen will likely be dissolved in the water. Alternatively, corn and/or bacteria may be added to the water to deplete the oxygen in the water. Also, oxygen reducing additives such as yeast, genetically altered bacteria known to those of skill in the arts of fermentation, or enzymes may be added to further deplete the oxygen levels which consume oxygen and sugars while producing carbon dioxide. In order to promote the depletion of oxygen levels, a secondary carbohydrate source, for instance corn, molasses, wheat or other sources of sugar, may be added to the water for use by the oxygen reducing additives. One benefit of the reduction of oxygen may be additional production of ethanol by the oxygen reducing additives.

The lack of sufficient oxygen in the water initiates the anaerobic process in the aquatic plants causing them to elongate and to produce ethanol. This may be encouraged by the introduction of chemical catalysts and CO₂. One chemical catalyst which may be included is 2,4-dichlorophenoxyacetic acid (known generically as 2,4d). Additional nutrients and salts such as salts of potassium, nitrogen and phosphorus may further be added to promote growth of the aquatic plants. Further, depending upon the species of aquatic plant being utilized, organic substrates, including but not limited to those such as sucrose, glucose and acetate, may also be added to the cell.

During the anaerobic process, the aquatic plants will increase in size dramatically and may achieve a lengthening of up to 10 times or more of its original length. The term ‘size’ here is to be understood to include a volume increase of plant matter which allows for it to store a larger amount of carbohydrates. This elongation provides cellular chambers for holding carbohydrates to be later formed by the aquatic plants. Additionally during the anaerobic process, ethanol is produced intra-cellularly and released extra-cellularly by the aquatic plants. This ethanol is then held within the water of the cell until it is removed by conventional methods. This step may take place from one to several days though in the case of Potamogeton pectinatus (or Stuckenia pectinata) a total of six days may suffice though longer periods, such as up to 14 days may be more beneficial to maximize output efficiencies. The time required will depend on many factors such as light diffusion, availability of nutrients, size of the cell, size of the plant and carbon content of the plant. The plant may be allowed to stay in anoxic conditions for up to several weeks. The determination of length of time is primarily dependent upon maximizing output of ethanol. When the plant decreases its ethanol production beyond useful parameters, there may be no need to retain it in the anoxic conditions.

During the anoxic period, the cell may be shielded from light sources which encourage photosynthesis. This lack of light encourages the release of the ethanol and prevents the formation of oxygen through photosynthesis. The light may be blocked by any conventional method to create dark conditions within the cell. It should also be understood that the term “light” which should be blocked only applies to those forms of radiation, or wavelengths of light, which act as a photosynthesis catalyst and is dependent upon the type of chemical receptors used by each plant. Therefore, the term “dark” as used herein is meant to denote the substantial absence of the frequencies of light which promote photosynthesis. This would allow, for instance, a cell to be placed within a dome or other structure which is illuminated by light visible to humans but which creates the “dark” condition for the plant.

The cell may be covered with one or more sealing barriers to prevent the unwanted introduction of oxygen into the cell and to better thermally control the cell. The sealing barrier may also be used to retain CO₂ within the cell, particularly if it is being added to the cell. Additionally, high N₂ levels may be maintained as well to further dilute any O₂ within the water or trapped between the seal and the cell. The sealing barrier would seal the cell to prevent fluid communication between the cell and the adjacent atmosphere. This will inhibit oxygen from entering the cell and will encourage the anaerobic process. The sealing barrier may be a translucent barrier to encourage the capturing of radiant heat from a light source which is either naturally and/or artificially used to provide light to the aquatic plants. The sealing barrier may or may not also constitute a light blocking barrier which is positioned on the cell to prevent light from entering the cell during the anaerobic process. The sealing and light blocking barriers may be made of conventional materials. However, it should be understood that a dwelling, tank, dome or other structure constructed around the cell may also define sealing and light block barriers should they be used in such a capacity.

It has been found that manipulating light and dark conditions can affect the manner in which the aquatic plants produce ethanol and sugars. For instance, some aquatic plants may be subjected to light for several continuous days defining a light phase followed by restriction to light for several continuous days defining a dark phase to better encourage the anaerobic, ethanol producing, process. One such plant, Stuckenia pectinata, has been shown to have a light phase for up to about 6 days after which its production of sugars levels off or reaches a predetermined optimal level. The term “day” is defined as 24 hours. This plant has a dark phase of between about 2 days and 30 days during which it may enter the anaerobic process and produce ethanol. Generally, the ratio of light phase to dark phase will be no more than 1:2 and as small as 1:10, with a more common ratio of between 1:2 and 1:7. It should be understood that during both of the light and dark phases, CO₂ may be added to the water to encourage both the formation of sugar and ethanol. Finally, the ability to control the light and dark phases above and the ratios described herein are not applicable to all aquatic plants as certain plants may experience ethanol production after less than 4 hours of dark phase. For these types of aquatic plants, the ratio of light phase to dark phase may be greater than 2:1, though such aquatic plants may have different limitations with respect to ethanol production than experienced with plants such as Stuckenia pectinata.

The next step is to expose the cell to light which stimulates photosynthesis and to stop the anaerobic process by allowing an oxygenated condition within the cell to initiate the aerobic process. This may be accomplished by introducing oxygenated water into the cell and by removing the anoxic water or allowing the water to oxygenate naturally by plant releasing of oxygen and exposure to an oxygenated atmosphere. During the aerobic process, as indicated above, the aquatic plants create carbohydrates through metabolic processes and then retain the carbohydrates within their elongated structures. Waste materials, such as waste biomass from the method 10, industrial waste, municipal waste and the like may be added to the cell to provide nutrients to the aquatic plants. Additionally, maximum sunlight/artificial light filtration is encouraged as is temperature regulation to promote growth of the aquatic plants. The light itself may be intensified by the addition of artificial light. Further, the pH of the cell must be monitored to prevent acidosis of the cell. This may be counteracted with calcium buffering compounds such as calcium carbonate and calcium chlorate, but will ultimately be dependent upon the tolerances of the particular aquatic plant species in the cell. The duration of the aerobic process is likewise dependent upon a number of factors but will typically end when carbohydrate production begins to slow or reaches a predetermined level. With Potamogeton pectinatus (Stuckenia pectinata) this may be between 2 days and 14 days depending upon environmental conditions within the cell.

Once maximum carbohydrate formation, or a predetermined level of such, is approached the oxygenated water is made anoxic to again begin the process of elongation and ethanol formation. The steps of creating anoxic conditions and oxygenated conditions are then repeated to continually promote elongation and ethanol production followed by carbohydrate production. This creates a self-sustaining cycle as the plant growth replenishes both plant matter lost to plant senescence and those plants which no longer meet established tolerances of ethanol production. Additional plant growth which cannot be used for replenishing purposes or for furnishing plant material for another cell may be removed and fermented using conventional methods to also produce ethanol. Carbon dioxide released during the fermentation process may be captured and returned to the cell to promote carbohydrate production. Plant waste, both before or after the fermentation process, may further be used for replenishing nutrients to the cell as feed material and/or may be processed for biochemical industrial usage such as in ethanol and diesel biofuels, pharmaceuticals, cosmetics, colorants, paints and the like.

Additional steps may be taken to increase plant growth and to further stimulate the production of ethanol. For instance, in order to increase ethanol formation and to prevent stagnation of the water, and eventual killing of the aquatic plants, a water agitation system may be incorporated to encourage the movement of water around and through the aquatic plants. This prevents the build up of ethanol and other plant waste materials adjacent to the plant and brings nutrients to the plant. It has been further found that agitation of the water promotes the suspension of water additives such as yeast. The agitation may include any form of wave movement through the plants or a sustained flow of water through the plants. Such a water movement system may be fluidly coupled to a circulation loop which removes the ethanol from the water after the water is piped or otherwise directed from the cell and before the water is returned to the cell. While the water is outside the cell in such a system, nutrients, antibiotics, O₂, CO₂, yeast or any other required or desired additives may be added to the water. Additionally, a circulation loop may be used to also remove the O₂ from the water to make the water anoxic before it is returned to the cell to create the anoxic condition.

It has also been found that by controlling the life cycles of the aquatic plants may be beneficial in lengthening the life spans of the aquatic plants. In particular, the life of some of the aquatic plants terminates after the flowering of those plants. This can be prevented by the cutting off of a top portion of the aquatic plants before they can flower. Such cutting will stop some of the aquatic plants from reaching the surface of the water and flowering. The plants may also be systemically cut and partially harvested to remove dead plant material and to thin the cell to allow for adequate light diffusion into the cell. The material cut may be allowed to remain in the cell to replenish nutrients to the cell.

While the method 20 is being practiced, bacterial and algal blooms may occur which can be controlled by antibiotics, bi-sulfates, hops, algaecides, chlorination, ultraviolet light exposure and other common practices. However, it has been discovered that that method 10 produces free carbohydrates, and in particular monosaccharides, which encourage bacterial growth within the cell. For this reason, it has been found to be beneficial to introduce ethanol producing yeasts into the cell for the purpose of decreasing the carbohydrate concentrations and inhibiting bacterial growth. Alternatively, or in conjunction with yeast, enzymes or bacterial may also be used to decrease carbohydrate concentrations. A beneficial outcome of the addition of yeast is an increase in ethanol output. As with the anaerobic process, the general equation for this process is C₆H₁₂O₆→2CO₂+2C₂H₅OH and is well known in the arts. The yeast may require replacement, particularly after the anoxic condition has been established and maintained for more than about three days, though this is dependent upon the strain of yeast being used.

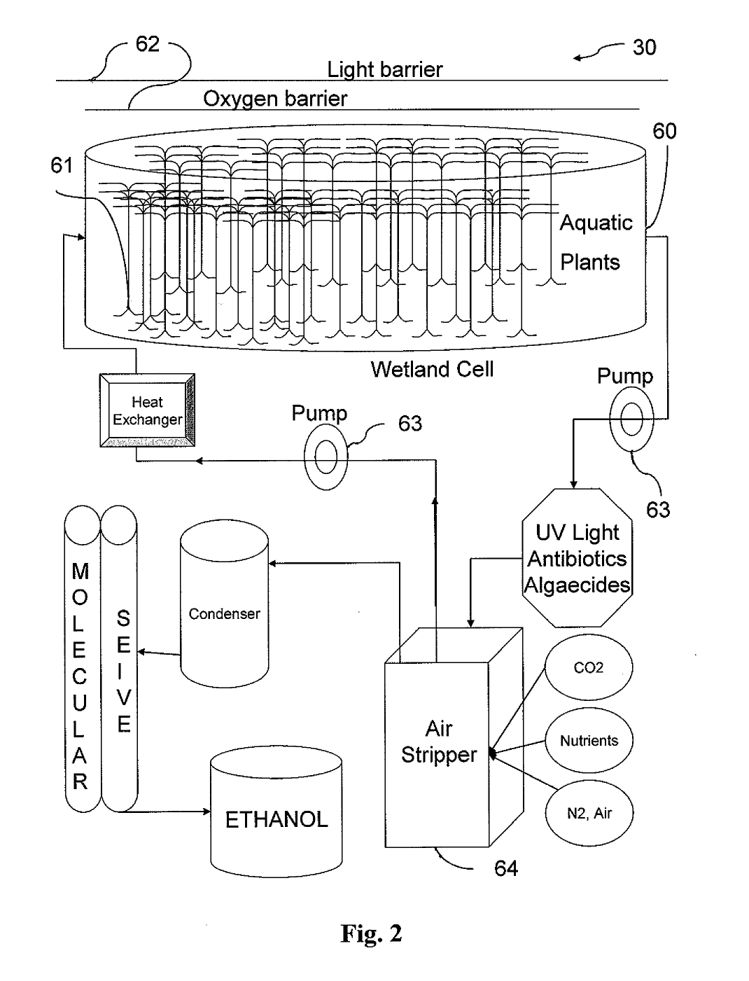

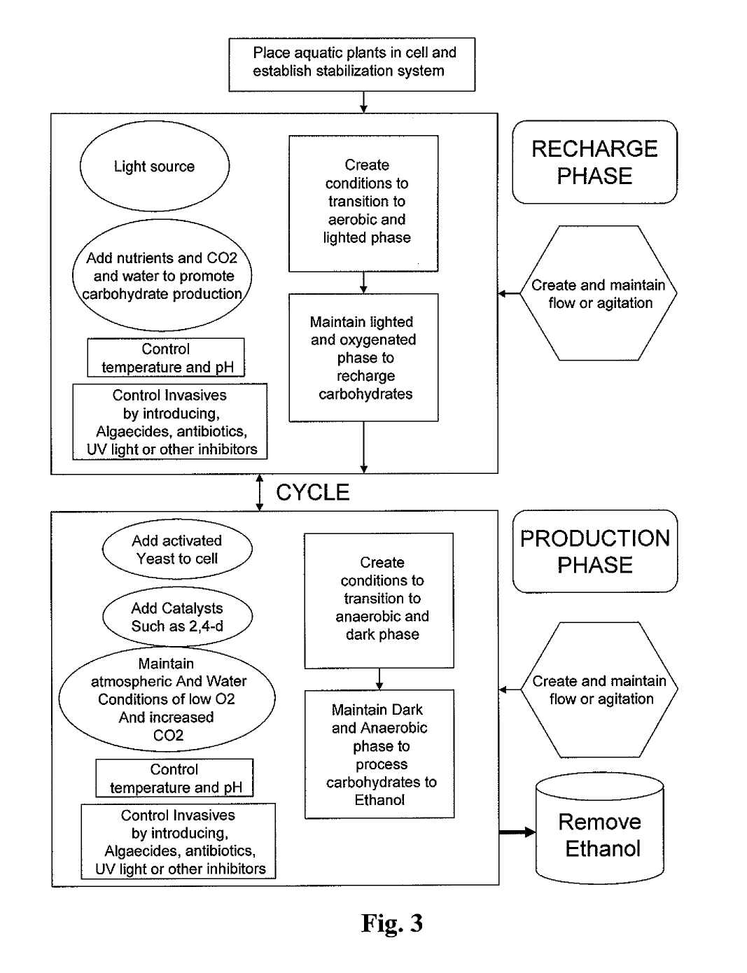

FIG. 2 depicts one method 30 particularly well suited for use in a single cell, though it should be understood that this method may also be used with multiple cells. FIG. 3 is a more detailed schematic view of FIG. 1 included to further explain the steps shown in FIG. 2. This method 30 also utilizes all concepts discussed above and generally includes the placement of the aquatic plants 61 in a cell 60. The cell 61 itself may be sunken into the ground surface or in a dwelling foundation, a partially sunken tank structure or a fully above ground tank structure. The cell 60 may have any particular shape, though a circular or loop type cell may be beneficial for encouraging the movement of water within the cell 60. The water may be moved in a conventional manner though one utilizing a gravity lift system may prove to be beneficial due to its lower power requirements.

The water is either oxygenated or allowed to become or remain oxygenated as light enters the cell during the light phase. Generally, the light phase is continued for between ½ day and 12 days, and more generally at least 3 to 6 days, to allow the aquatic plants 61 to form sugars, though this time frame may be adjusted for plant specific requirements. The sealing barrier 62, which may include separable light and air barriers so that the light barrier alone is removed, may be used at this time to conserve heat should such be necessary to obtain an optimal temperature for the particular aquatic plant 61 or plants being used. The sealing barrier 62 may also be used to maintain high carbon dioxide levels in the water to for carbohydrate production. After the termination of the light phase, the dark phase is initiated and the water is made anoxic to encourage the anaerobic process. When the light phase ends, there is a transition period between the oxygenated phase and the anoxic phase where the amount of oxygen is being depleted. During the transition period, it may be beneficial to add the yeast to the cell which will stimulate the reduction of the oxygen and will allow the yeast to produce the ethanol. The ethanol formed by the yeast may act as a catalyst for anaerobic activity by the plant and will offer an additional ethanol production outlet.

Once ethanol production drops to limits which are no longer efficient or the stress on the plants 61 becomes too great, the cell 60 is again exposed to light and is allowed to become, or made, oxygenated. This three part cycle may more broadly be defined to include: 1) a recharge phase wherein the water is oxygenated and the plant is exposed to light so that carbohydrates are formed, 2) a transition phase wherein the water is being made anoxic, the cell is deprived of photosynthesis inducing light and yeast may be added to form ethanol and deplete oxygen, and 3) an anoxic phase wherein the plant enters an anaerobic process of releasing ethanol. A fourth phase may be defined as a second transition phase wherein the water is again allowed to become oxygenated. The phases may each be modified as taught herein to maximize plant growth and ethanol output. In one method, the recharge phase may occur over 0.5-12 days, followed by 0.5-6 days of the transition phase, which is then followed by at least 6 days of anoxic phase which may be increased to more than 20 days depending on the type of plant being utilized. In another method, the recharge phase may occur over 3-6 days, followed by 2-6 days of the transition phase, which is then followed by at least 6 days of anoxic phase which may be increased to more than 20 days depending on the type of plant being utilized.

During the above light and dark phases, the water may be pulled out from and reintroduced into the cell through a closed loop system, such as by way of pumps 63, which may include an access point to the water to allow all additives discussed above to be supplied to the water without over exposing the water to the atmosphere. The closed loop system may further include an ethanol removal assembly such as, but not limited to, conventional air strippers 64. This will allow the ethanol to be removed continuously while leaving the light blocking barrier and sealing barrier 62 in place. The air stripper 64 may be utilized to allow for the introduction of CO₂, Nitrogen and nutrients into the water as well. The air stripper 64 is fluidly coupled to a condenser and molecular sieve to concentrate the ethanol as is well known in the art. While the water is removed, it may be exposed to ultraviolet light and/or antibiotics and algaecides may be added to maintain a healthy cell 60 free of unwanted bacterial and algae growths.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure.

I claim:

1. A method of inducing formation of ethanol, said method comprising the steps of:
placing aquatic plants in a cell containing water;
initiating a recharge phase wherein said water is oxygenated to define an oxygenated condition and said plants are exposed to light to define a light phase to encourage carbohydrate formation though aerobic metabolism;
instigating a transition phase wherein said water is made anoxic to define an anoxic condition and said cell is deprived of photosynthesis inducing light to define a dark phase;
encouraging an anoxic phase by retaining said plants in said anoxic condition and in said dark phase to encourage anaerobic metabolic in said plants such that said plants release ethanol into said water; and
capturing ethanol released into said water by said plants.

2. The method according to claim 1, wherein the step of instigating a transition phase further includes the step of adding yeast to said water.

3. The method according to claim 1, wherein said recharge phase is maintained for between 0.5 day and 12 days, said transition phase is maintained between 0.5 days and 6 days and said anoxic phase is maintained for at least 3 days.

4. The method according to claim 1, further including the step of repeating the steps of initiating a recharge phase, instigating a transition phase and encouraging an anoxic phase to stimulate release of ethanol.

5. The method according to claim 1, further including the step covering said cell with a light blocking cover during the anoxic condition to inhibit light from entering said cell to define said dark phase.

6. The method according to claim 5, wherein said dark phase is continuous for at least 2 days, said light phase having a duration being less than a 1:2 ratio with respect to said dark phase.

7. The method according to claim 1, further including the step of introducing catalysts to increase anaerobic metabolism.

8. The method of claim 1, further including the step of creating water agitation within said cell to prevent buildup of plant waste materials adjacent to the aquatic plants during said anoxic condition.

9. The method of claim 4, further including the step of creating water agitation within said cell to prevent buildup of plant waste materials adjacent to the aquatic plants during said anoxic condition.

10. The method of claim 1, wherein the step of placing aquatic plants in a cell includes said aquatic plants being selected from the family Potamogetonaceae.

11. The method according to claim 5, wherein said dark phase is continuous for at least 2 days, said light phase having a duration necessary to replace lost carbohydrate content necessary to maintain said method.

12. The method according to claim 1, wherein said recharge phase is maintained to reestablish depleted carbohydrates lost during said anoxic phase, said transition phase is maintained between 2 days and 6 days and said anoxic phase is maintained for at least 6 days.

13. The method according to claim 1, wherein said light phase has a duration being less than a 1:2 ratio with respect to said dark phase.

14. The method according to claim 2, further including the step of adding a carbohydrate source during said transition phase to promote anoxic conditions.

15. The method according to claim 1, wherein the step of instigating a transition phase further includes the step of adding an oxygen reducing yeast, bacteria or enzyme to said water.

16. The method according to claim 15, further including the step of adding a carbohydrate source during said transition phase to promote anoxic conditions.
